(19) United States
(12) Patent Application Publication
(10) Pub. No.: US 2008/0008734 A1
(43) Pub. Date: Jan. 10, 2008

(54) Reduction of skin irritation caused by biomedical electrodes

(76) Inventors: James J. Perrault, Vista, CA (US); Solomon Shenkute, Escondido, CA (US); Svitlana Kroll, Riverside, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/481,126

(22) Filed: Jul. 5, 2006

(51) Int. Cl. A61N 1/00

(52) U.S. Cl. 424/423; 514/474; 607/2

(57) ABSTRACT

A method and/or composition for monitoring electrical signals from a patient through a biomedical electrode or delivering an effective amount of electrical stimulation to said patient without causing erythema which comprises providing an antioxidant to the skin of said patient via solution, lotion, or gel prior to adhering said biomedical electrode thereto or incorporating in the hydrogel conductive adhesive of the electrode for antioxidant activity during electrical contact. Chemical precursors of some antioxidants are utilized for the manufacture and stable incorporation into hydrogel conductive adhesives to avoid interference with the preparation of the conductive adhesive by free radical polymerization.

### FIELD OF INVENTION

The present invention relates to compositions and methods for alleviating and/or preventing oxidative skin damage resulting from monitoring of a patient with a bioelectrode or electrical stimulation of a patient with transcutaneous electrodes.

### BACKGROUND OF THE ART

Throughout the history of NMES (neuromuscular electrical stimulation ), skin irritation been attributed to chemical and thermal causes (Fischer, Cutis 21: 24-47 (1978); Zugerman, J Am Acad Derm 6(5): 936-9 (1982); Lambert et al, Proceedings of IEEE-EMBEC and CMBEC, 647-8 (1995)). As the chemical compositions of coupling gels improved to use generally-recognized-as-safe (“GRAS”) ingredients, the electrolysis of water into acid and oxygen gas at the anode and base and hydrogen gas at the cathode was identified (Leeming et al, JAMA, 214(9): 1681-4 (1970); Cooper et al, Anesthesiology 75: 168-172 (1990);Yosida and Patriciu, Proceedings of the 5^{th} Annual Conference of the International Functional Electrical Stimulation Society, 282-4 (2000)). In cases of prolonged net DC (direct current) stimulation and/or compromised skin, the generation of base (OH⁻) at the cathode leads to permanent skin damage. U.S. Pat. Nos. 4,744,787; 4,973,303; 5,624,415; 6,347,246 and others addressed this by incorporating buffering systems. Yet, there still remained transient erythema, even in the case of balanced biphasic waveforms with no net DC, where erythema occurred (Leger, Advanced Drug Delivery Reviews 9: 289-307 (1992)) at both electrodes and remained unexplained and unaddressed.

It is known that reactive oxygen species are produced by the incomplete reduction of oxygen. The complete reduction of one molecule of O₂ to water is a four electron process. A one electron reduction of O₂ yields superoxide anion O₂.⁻, reduction by an additional electron yields hydrogen peroxide H₂O₂, and reduction by a third electron yields a hydroxyl radical OH and hydroxide ion OH⁻. This series of reactions is as follows:

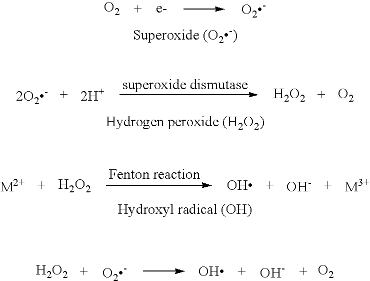

Electrical stimulation generates partially reduced species of oxygen from both entrained air and carboxy materials in the electrode components at the interface between the conductor and the skin coupling hydrogel adhesive; Common carboxy groups in hydrogel adhesives are alcohols R—OH, amides R—CON—R, carboxylic acids R—COOH, esters R—COO—R, ethers R—O—R, and ketones R—CO—R; where R is a substituted or unsubstituted hydrocarbon chain. (Substituted hydrocarbons may be substituted with heteroatoms selected from the group consisting of oxygen, sulfur, phosphorous, and halogen atoms.) Examples of possible reactive oxygen species generated by electrolysis at the conductor-adhesive interface include atomic oxygen O, singlet oxygen O., superoxide anion O₂.⁻, hydrogen peroxide H₂O₂, hydroxyradical OH., alkoxy radicals R—O., alkyl radicals R., and alkylperoxy radicals R—O—O. Because some of these reactive oxygen species have one unpaired electron in their molecular orbitals, they are also commonly known as free radicals. Free radicals have potent oxidating effect which is the basis for their destructive effects on lipids, proteins, nucleic acids and the extracellular matrix of the skin. The quantity of reactive oxygen species generated by electrical stimulation is small but detectable with a peroxide test kit which measures such species in the range of 20-200 ppm with a detection limit of 0.5 ppm.

In U.S. Pat. No. 5,972,993, Ptchelintsev discloses that rosacea and sensitive skin may be treatable with antioxidants. In U.S. Pat. No. 6,344,214, Lorenz discloses that the very potent antioxidant, Astaxathin, retards, ameliorates and prevents cold sores, canker sores, and the damage of UVA radiation in sunlight. An antioxidant is a molecule that can react with reactive oxygen species and negate their chemical reactivity and/or can prevent generation of free radical species from non-radical oxidative species such as hydrogen peroxide. For example, a compound that has a phenolic hydroxyl group such as MEHQ (monomethyl ether of hydroquinone) can react with free radicals and form phenoxyradicals which are significantly less reactive and less damaging to biological systems because the energy of phenoxyradicals is lower due to the delocalization of the unpaired electron around their aromatic ring. This reaction is illustrated below:

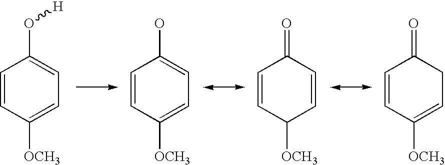

Other common modes of action for antioxidants are trapping and chelating the reactive oxygen species.

While various chemicals exhibit antioxidant properties, the necessary concentration of any antioxidant in topical formulation will vary depending upon the specific antioxidant used and its relative potency. Also, various antioxidants exhibit negative and undesired effects at different relative concentrations; biological and otherwise.

For example, it was found that the inclusion of various antioxidants into conductive hydrogel adhesives used for coupling of an electrode to the skin in skin surface electrodes caused certain undesirable effects. In the majority of conductive hydrogel adhesives used in these biomedical electrodes are UV cured acrylics formed by the free radical polymerization of hydrophilic acrylic monomers in water. UV light generates free radicals from photoinitiators, however antioxidants react with these free radicals thereby inhibiting the polymerization of the acrylic monomers. Antioxidants such as MEHQ are used to preserve acrylic monomers until use. It was found that therapeutic amounts of antioxidants were very detrimental to the degree of free radical polymerization needed to provide a suitable adhesive for a biomedical electrode. A few ppm of the very potent antioxidant Astaxathin inhibited polymerization. The greater than 1500 ppm of MEHQ alone that was needed to prevent and/or alleviate stimulation erythema gave a very weak hydrogel adhesive that cohesively failed leaving residue on the skin.

In view of the aforementioned problems with skin irritation caused by the bioelectrodes, including the transcutaneous electrodes of the art, it is one object of this invention is to alleviate and/or prevent skin damage or erythema resulting from the use of topically-adhered, biomedical electrodes to monitor and/or stimulate a patient.

It is a further object of this invention to provide an adhesive for a biomedical electrode that incorporates an antioxidant to prevent erythema while having suitable adhesion to the skin of a patient undergoing treatment.

### SUMMARY OF THE INVENTION

It has now been found that the effect of the free radicals and other reactive oxygen species, as described above, on skin is treatable with antioxidants.

Antioxidants can donate electrons without generating potentially harmful chain reactions and oxidation of cellular components, and thus provide protection from oxidative damage. Peroxides can be found in some skin contact adhesives without generation by electrolysis so the addition of antioxidants further enhances biocompatibility.

In addition, the objects of this invention are achieved by including an antioxidant in a free radical polymerizable monomer mixture which is polymerized, in the presence of an ultraviolet (UV) sensitive photoinitiator which photoinitiator reacts with UV light to generate free radicals, to provide a solid, adhesive interface for an electrode, suitable for electrical contact with a patient's skin, wherein said antioxidant is in the form of a prodrug which does not react with said free radicals during polymerization, but upon contact of said prodrug with the patient's skin it is converted to said antioxidant. For the purpose of describing the present invention, a prodrug is a chemical moiety that is not an active agent until the body has acted upon it in a way that converts it to the active form.

Preferably, said prodrug is a chemical moiety that is converted into a vitamin, e.g. Vitamin A, C or E, and more preferably said prodrug is converted into a water-soluble vitamin, i.e. Vitamin C. It has been found that Sodium Ascorbyl Phosphate (SAP), a prodrug form of Vitamin C, is especially suitable as an antioxidant to prevent erythema of the skin yet does not inhibit free radical polymerization of a free radical-polymerizable monomer mixture that is suitable for polymerization to provide an adhesive for a biomedical electrode. Ascorbic Acid, i.e. Vitamin C, itself, did significantly inhibit UV initiated free radical polymerization of such monomer mixture. Sodium Ascorbyl Phosphate (SAP), shown below, is cleaved enzymatically by phosphatase enzyme in the skin to release active Vitamin C.

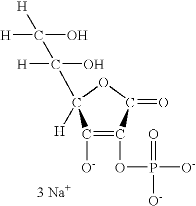

However, it was found that SAP was not stable over time in the common pH 4.0±0.5 aqueous environment of the hydrogel adhesives that are preferred for providing a solid, conductive adhesive interface for a biomedical electrode. (See U.S. Pat. No. 6,842,636 B2 to Perrault, et al. and U.S. Pat. No. 6,115,625 to Heard et al.).

In U.S. Pat. No. 6,774,247, Behnam disclosed the stabilization of an aqueous solution of ascorbic acid with emulsifiers having HLB (hydrophilic-lipophilic balance ) values between 9-18 such as polysorbate. It has now been found that Polysorbate 20 is the most compatible with hydrogel adhesives and may be used to stabilize the SAP that is incorporated into the monomer mixtures that are precursor to the solid adhesive interface of the biomedical electrodes of this invention.

In addition, additional antioxidants may be added to delay oxidative damage to the SAP, itself, either during the polymerization of the monomer mixture or the use of the resulting biomedical electrode. Therefore, the topical antioxidant compositions of this invention may contain a single antioxidant, or a combination of antioxidants, i.e. an antioxidant blend. The term “antioxidant” as used herein is intended to encompass both single antioxidant as well as antioxidant blends. It is also contemplated that the final composition contains a total concentration of antioxidant, whether as a single antioxidant or as antioxidant blends, that ranges from about 0.01 wt % to about 10.0 wt % dependent on potency and manufacturing concerns such as may effect UV curing of the monomer mixture.

### BRIEF DESCRIPTION OF THE DRAWINGS

FIG. 1 shows a perspective view of the electrode of the invention.

FIG. 2 shows a cross section inside through the electrode of FIG. 1.

FIG. 3 shows the erythema after 40 minutes of NMES stimulation through polyvinylpyrrolidone conductive adhesive electrodes with and without prior antioxidant treatment of the skin.

FIG. 4 shows erythema after 40 minutes of NMES stimulation through the acrylic hydrogel adhesive of example 7 alongside a similar acrylic adhesive without antioxidants. Example 7 was nine months old at the time of this test and photograph.

### DETAILED DESCRIPTION OF THE INVENTION

In particular, the present invention provides an electrode suitable for providing electrical contact with a patent's skin which electrode includes a conductive member adapted for connection to an external electrical apparatus and a non-liquid or solid water-containing film for electrically interfacing to said patent's skin, said non-liquid film being electrically and mechanically connected to said conductive member. The non-liquid water-containing film includes an electrically conductive organic polymer derived from a monomer mixture which undergoes free radical catalyzed polymerization in the presence of UV light and a photoinitiator to provide said organic polymer, wherein said monomer mixture further comprises a chemical moiety, which is a precursor of an antioxidant, which chemical moiety does not react with the free radicals derived from said photoinitiator under polymerization conditions but, upon contact with the skin of a patient, is converted into said antioxidant. Said antioxidant is preferably a vitamin, e.g. Vitamin A, C, or E, and more preferably said vitamin is a water-soluble vitamin, e.g. Vitamin C.

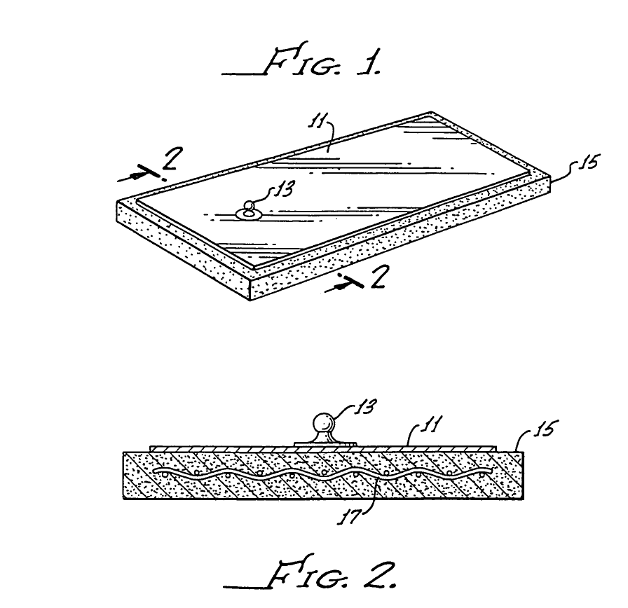

The electrode configuration is shown in FIG. 1. A conductive member 11 is cut, stamped or otherwise shaped out of a piece of conductive material which may be metal foil or a polyester, film, e.g., a polyethylene terephtalate (Mylar®) coated with a metal layer or contiguous metallic ink print or a carbon impregnated plastic or elastomer sometimes printed with a metal ink to enhance conductivity. The shape to which this conductive member 11 is formed will depend upon the particular application in which it is to be used. The shape is sometimes round but may be as shown in FIG. 1, rectangularly shaped.

Alternately, other metallic foils, conductive polymers, graphitized or metalized cloth or wire mesh may be used as the conductive member. In particular, the knit conductive fabric disclosed in U.S. Pat. No. 4,722,354 to Axelgaard, et al. may be utilized as the conductive member. For each material, an appropriate strength and thickness is to be chosen to yield a pliable, yet sufficiently strong member 11. When the conductive member 11 is of metal foil, it usually is of 1-10 mil thickness.

Secured to the outer surface of the conductive member 11 is a connector 13 for providing a medium to which external signal cables may be attached for electrically communicating with the conductive member 11. This connector 13 may be a conductive swaged snap fastener 13, as shown in the accompanying drawings, which is available commercially. This fastener 13 is mechanically and electrically attached to the conductive member 11, extending perpendicularly from the outer surface of this member 11. Alternatively, when the conductive member is a knit conductive fabric, the electrical connector may be stranded stainless steel as shown in U.S. Pat. No. 4,722,354.

Abutting the inner surface of the conductive member 11 is an electrically conductive skin-interface substrate 15. This substrate 15 is a layer of material being typically a film or sheet which will be described below.

The conductive substrate 15 is shaped correspondingly to the conductive member 11. When constructed in combination with a rectangular member 11, the substrate 15 is also rectangular. The film thickness of this substrate 15 is uniform throughout, however, this uniform film may be of various thicknesses. A range of 10 to 100 mils, e.g., about 50 mils is common.

As will be discussed below, the substrate 15 is a film or sheet having adhesive properties, thus when it is brought into contact with the conductive member 11, it will adhere to that member 11 providing electrical connection with it.

A supporting scrim 17, FIG. 2 may be used in electrode configurations where a greater thickness substrate 15 film is used. This scrim 17, while not a necessary part of the electrode, will tend to support by being distributed throughout the substrate 15. A further advantage to the use of this scrim 17 is that it acts to reinforce and strengthen the substrate 15.

The scrim 17 is positioned within and through the thickness of the substrate 15, in alignment with the conductive member 11, and is of a size to extend completely under the conductive member 11. The scrim 17 can be a woven or non-woven spun-bonded polyester fabric, a net of a stretched, embossed melt-extruded polymeric film, a sheet of polyolefin monofilaments heat-sealed together at their interstices, a thin sheet of a thermoplastic polymer with holes heat-stamped in a geometric pattern or any other supportive media. The scrim 17 may be any material allowing transmission of light for curing if only cured from one side. Also, the scrim 17 should retain most of its strength when wetted by the monomer mixture.

Suitable monomer mixtures for use in the practice of the present invention are found in U.S. Pat. No. 6,842,636 to Perrault, et al. and U.S. Pat. No. 6,115,625 to Heard, et al. both of which are hereby incorporated in their entirety for the description of the various suitable monomer mixtures, the polymerization thereof and the fabrication and resulting structure of the medical electrodes of this invention.

The electrically conductive organic polymers that are utilized in preparing the conductive adhesive may be derived from the polymerization of the water soluble monomer mixture, e.g. an acrylic acid or methacrylic acid containing monomer mixture and preferably by the copolymerization of a mixture of monomer acrylic acid and monomers glycolvinylether and/or vinylpyrrolidone. (Metha)crylics are electron accepting monomers and glycol vinyl ether and vinylpyrrolidone are electron donating monomers. Said organic polymer may comprise 25 to 75 parts per hundred, by weight (pph), e.g., 30 to 60 pph, acrylic acid and 75 to 25 pph, e.g. 70 to 40 pph, of a glycolvinylether and/or vinylpyrrolidone. In addition, the above mixture of comonomers, the organic polymer, may further include additional comonomers; in particular, the acrylic acid may be completely or partially replaced with AMPS.

Preferably, the glycolvinylether may be selected from the group consisting of hydroxybutyl vinyl ether, ethyleneglycolvinylether, diethyleneglycolmonovinylether, and triethyleneglycolmethylvinylether. Most preferably the glycolvinyl ether is diethylene glycol monovinyl ether.

Furthermore, the organic polymer may comprise about 0.01 to 1.5 pph of a crosslinking agent, such as methylene bisacrylamide, to increase the molecular weight and cohesivity of the conductive organic polymer through crosslinking. Other comonomers having at least two copolymerizable olefinic moieties, especially difunctional or trifunctional derivatives of acrylic acids, may be utilized. For example, polyethylene glycol dimethacrylates and diacrylates having a molecular weight of from about 200 to about 600 and ethoxylated trimethlolpropane triacrylate (ETMPTA) are preferred crosslinking agents.

The comonomer mixture that is copolymerized to provide the conductive organic polymer may also include a polyhydric alcohol, e.g. polyhydroxyhydrocarbons and oxyalkyls, e.g., polyetheneglycol, sorbitol, glycerol, etc. to plasticize the organic polymer. The polyhydric functions as a humecant, i.e., it absorbs moisture and promotes conductivity of the adhesive. The polyhydric alcohol may comprise from 25 to 75 pph, preferably from 40 to 60 pph, e.g., about 37 to 53 pph of the comonomer mixture. Most preferably, the polyhydric alcohol is glycerol.

The comonomer mixture that is copolymerized to provide the conductive organic polymer may also include a thickening agent. The thickening agent may be a high molecular weight polymer or copolymer such as a N-vinylpyrrolidone/vinylacetate copolymer (Luviskol VA 73W or VA 64W) available from BASF; methylvinylether/maleic anhybride copolymer (Gantrez® S95), which is available from ISP; ethylene/maleic anhydride (EMA) Copolymer, which is available from Zeeland Chemical; and N-vinylpyrrolidone/acrylic acid Acrylidone® (ACP-1041 or Acrylidone 1005), which is available from ISP, and may comprise from about 0.5 to 8 pph of the comonomer mixture, e.g. about 2 to 5 pph. The N-vinyl pyrrolidone/vinylacetate copolymer disclosed above is especially preferred for use in the compositions of this invention.

Magnesium acetate may also be included when the electrode is utilized as a stimulating electrode. Potassium chloride may be added when the electrode is utilized as a sensing electrode.

The above comonomer mixture is copolymerized or cured by thermal or ultraviolet (UV) radiation to provide the necessary free radicals. Therefore, an ultraviolet sensitive curing agent is provided in the comonomer mixture at a concentration of from 0.05 to 3 pph, preferably from 0.5 to 2.0 pph. Suitable curing agents are 2-hydroxy-2 methyl-1-phenyl-propan-2-one (available as Darocur 1173®), 4-(2-hydroxyethoxy)phenyl(2-hydroxy-2-phenyl(2-hydroxy-2-propyl)ketone (available as Darocur 2959®), 2,2-dimethoxy-2-phenyl acetophenone (available as Irgacure® 651), 1-[4-(2-Hydroxyethoxy)-phenyl]-2-hydroxy-2-methyl-1-propane-1-one (available as Irgacure® 2959) or 1-hydroxycyclohexylphenylketone (available as Irgacure 184), all of which are available from Ciba-Geigy and trimethyl benzoyl diphenyl phosphine oxide (available as Esacure DP250 from Lamberti).

Thus, to prepare conductive adhesive, the following gelled comonomer mixtures may be subjected to chemical, thermal or ultraviolet polymerization conditions:

| | Broad | Preferred |
|---|---|---|
| Ingredient | Range pph | Range pph |
| Acrylic acid | 2–20 | 4–12 |
| Electron donor co-monomer | 2–20 | 3–10 |
| Crosslinker | 0.01–3 | 0.01–2.0 |
| Thickener | 0–8 | 0–3.0 |
| Glycerin | 25–75 | 35–60 |
| UV sensitive curing agents | 0.5–3 | 0.5–1.5 |
| Distilled water | 10–40 | 15–30 |

The acrylic acid is preferably partially neutralized with a basic potassium or sodium oxide, hydroxide, or carbonate or amine, e.g. triethanolamine. For example, from 25 to 75 molar percent acrylic acid may be neutralized.

A buffer may also be included in the comonomer mixture, e.g. from 0.2 to 2 pph of potassium sodium tartrate, or aluminum potassium sulfate (a further function of the AL⁺³ ion of the above buffer and Mg⁺² ions, as well, is that such ions function as firming agents for compositions of this invention).

As previously discussed, the medical electrodes of the prior art, even the improved electrodes described in U.S. Pat. No. 6,115,625 and U.S. Pat. No. 6,842,636 may cause erythema under normal conditions of use, e.g. for stimulation and/or monitoring, whereby said electrode is adhered to the skin of a patient. Thus, the present invention provides a suitable antioxidant to prevent or alleviate the erythema resulting from the generation of free radicals, as discussed above, under normal conditions of use. Suitable antioxidants may be selected from known antioxidants that interact with the above free radical oxygen moieties described above to convert said free radicals into innocuous compounds, e.g. water, etc.

In Particular, said Antioxidants may be Selected from the Following Groups:

Group I. Phenolic compounds that contain one or more hydroxyl groups (—OH) connected directly to a benzene ring and to another unsaturated chemical grouping. Preferred examples of such compounds include hydroquinone, butylated hydroxyl toluene, butylated hydroxyl anisole, MEHQ, chlorogenic acid, caffeoylquinic acid, cinnamoylquinic acid, glabridin (also known as “PT-40”), bioflavonoids of flavone, isoflavone and flavonol structural type in both glycosilated and non-glycosilated forms, camosic acid (also known as “rosemary extract” or “oxyless R”), naringenin, naringin, hesperetin, hesperedin, citrus bioflavenoid complexes, quercetin, rutin, ellagic acid, tocopherols and their derivatives (for example, Vitamin E acetate), ascorbic acid and its derivatives (for example Sodium Ascorbyl Phosphate), propyl gallate, sylibin (also known as “silymarin”), cycloartenyl ferrulate, gamma-oryzanol, benzotriazole, tolyltriazole, Irganox 1010, and caffeic acid.

Group II. Sulfur-containing compounds that contain one or more sulfhydryl groups (—SH) or one or more disulfide groups (—S—S—). Preferred examples of such sulfhydryl compounds include glutathione, cysteine, N-acetyl cysteine, cysteine hydrochloride, alpha-lipoic acid, dihydrolipoic acid, thiolactic acid, sodium sulfite, sodium metabisulfite, sodium bisulfite, sodium thiosulfite, sodium formaldehyde sulfoxylate, thioglycerol, thiosorbitol, thiourea, thioglycolic acid, and zinc dithiophosphate.

Group III. Polyene compounds that have conjugated systems of double bonds. Preferred examples of such polyene compounds include sorbic acid, carotenoids, beta-carotene, lycopene, retinoids (for example, Vitamin A alcohol), Astaxanthin, and lutein.

Minerals such as selenium and manganese can also be efficacious in combating the tissue damage caused by reactive oxygen species. Synthetic antioxidants which are inherently skin irritants such as hexamethylene tetramine are not to be considered.

As further noted above, said antioxidants may interfere with the free radical catalyzed polymerization of the monomer mixture that is utilized to prepare the conductive adhesive for the above medical electrodes. Thus, the antioxidant is provided in the form of a compound that is inactive as regarding its ability to interfere with the above free radical polymerization but is converted upon contact with the skin of a patient, over time, into the antioxidant.

As will be appreciated, many of the above antioxidants have functional groups which can be conveniently converted into forms wherein the resulting molecule will not interfere with the free radical polymerization process but in the presence of endogenous enzymes will be “reconverted” into the active antioxidant.

For example, antioxidants having hydroxyl, carboxylic acid or phosphate functional groups can be converted into esters which do not interfere with the free radical polymerization process, but will be converted by an endogenous hydrolase and/or phosphatase into the active antioxidant.

The antioxidant will be provided in an amount sufficient to prevent and/or alleviate erythema under normal conditions of use. For example, the antioxidant, in the form of a antioxidant precursor which is a chemical moiety which does not interfere with the free radical polymerization of the monomer mixture but upon contact with the skin of a patient converts to said antioxidant, will comprise from 0.01 to 10%, by weight, of the monomer mixture, preferably from 0.20 to 3.0%, by weight of said mixture.

As stated above, the antioxidant is preferably a vitamin and, more preferably, a water-soluble vitamin such as Vitamin C. Most preferably, said antioxidant precursor is sodium ascorbyl phosphate.

When the antioxidant is a vitamin, the precursor may be defined as a prodrug. Prodrugs of the above antioxidants are well known in the art and include the following:

Sodium ascorbyl phosphate, magnesium ascorbyl phosphate, sodium ascorbyl 2-phosphate 6-palmitate, disodium ascorbyl sulfate, magnesium boroascorbate, ascorbyl tetraisopalmitate, tocopheryl acetate, tocopheryl succinate, tocopherol 1000 PEG succinate, tocopheryl phosphate, delta-tocopherol glucoside, tocopheryl nicotinate, retinyl acetate, retinyl palmitate, caffeic acid phenetyl ester, octyl caffeate, magnesium thiosulfate, zinc dithiophosphate, and selenium proteinate.

The monomer mixture, including the prodrug, may be further stabilized against interference with the free radical catalyzed polymerization process by the prodrug and/or any antioxidant, prematurely derived therefrom, by the incorporation of a suitable nonionic surfactant. An especially suitable class of nonionic surfactants are the polyoxy ethylene adducts of fatty acids, e.g. polyoxyethylene sorbitan monooleates. These surfactants are sold under the conventional name of Polysorbates.

The invention is further illustrated by the following examples which are illustrative of a specific mode of practicing the invention and is not intended as limiting the scope of the appended claims.

### EXAMPLE 1

A solution of 2.0% (by weight) Sodium Ascorbyl Phosphate in deionized water was prepared with 2.0% Polysorbate 20 and 0.1% MEHQ added for stability. The solution was applied by laboratory swab to the skin sites of 1 of 2 each skin electrodes to be tested. The electrodes were constructed of an electron beam crosslinked polyvinylpyrrolidone hydrogel conductive adhesive, stainless steel wire—polyester knit fabric conductor, and standard NMES leadwire.

The stimulation was done with an EMPI Focus stimulator through subject electrodes for 40 minutes. The setting on the stimulator was a balanced biphasic waveform of 40 milliamp amplitude, 35 Hz frequency, and 200 microsecond pulse width with an On/Off duty cycle of 7 and 12 seconds respectively.

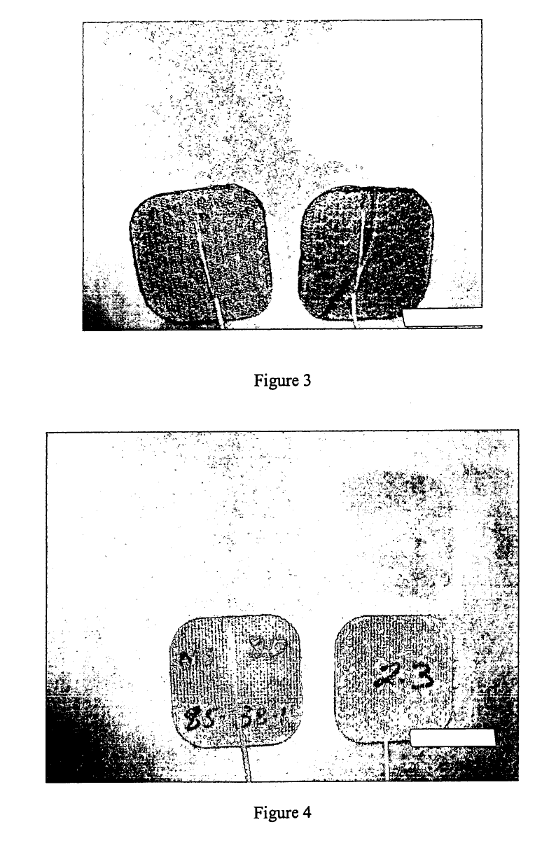

When stimulation was completed and electrodes removed, typical stimulation erythema was observed on the untreated skin. This transient erythema remained for over an hour. The treated skin had very little erythema and this was resolved in a matter of minutes. These results are shown in FIG. 3.

### EXAMPLE 2

An antioxidant lotion was prepared from an example formulation in the BASF Technical Bulletin for Sodium Ascorbyl Phosphate dated January 2003. It was denoted as BASF 53/00186NDE. The active ingredients were 1.0% Sodium Ascorbyl Phosphate and 0.5% Tocopheryl (Vitamin E) Acetate.

TABLE 1

| | % | Ingredient | INCl Name |
|---|---|---|---|
| A | 6.00 | Cremophor ® WO7 | PEG-7 Hydrogenated |
| | | | Castor Oil |
| | 0.50 | Cremophor ® RH 410 | PEG-40 Hydrogenated |
| | | | Castor Oil |
| | 7.00 | Isopropyl Palmitate | Isopropyl Palmitate |
| | 2.00 | Elfacos ® ST 9 | PEG-45/Dodecyl Glycol |
| | | | Copolymer |
| | 3.00 | Jojoba Oil | Jojoba (Buxus Chinesis) |
| | | | Oil |
| | 0.60 | Magnesium stearate | Magnesium Stearate |
| | 8.00 | Uvinul ® MC 80 | Ethylhexyl |
| | | | Methoxycinnamate |
| | 5.00 | Finsolv TN | C12–15 Alkyl Benzoate |
| | 4.00 | Kemira 160 | Titanium Dioxide |
| B | 5.00 | 1,2-Propylene Glycol Care | Propylene Glycol |
| | 0.20 | Edeta ® BD | EDTA |
| | 57.20 | Water, dist. | Aqua |
| C | 0.50 | Vitamin E Acetate | Tocopheryl Acetate |
| | 1.00 | Sodium Ascorbyl | Sodium Ascorby |
| | | Phosphate | Phosphate |

### EXAMPLES 3 THRU 8

Conductive hydrogel adhesives were UV cured from the formulas in Table 1 below. The coatings were 35 mil (0.035 inch) thick and were made in 2 inch by 2 inch electrodes with carbon filled PVC film conductor and standard NMES leadwires.

TABLE 2

| | EXAMPLE | | | | | |
|---|---|---|---|---|---|---|
| FORMULATION | 3 | 4 | 5 | 6 | 7 | 8 |
| COMPONENT LIST | | | | | | |
| Deionized Water | 15.044% | 16.500% | 21.450% | 15.910% | 12.953% | 13.797% |
| Potassium Chloride | 0.250% | | | 0.800% | 2.000% | 2.00% |
| Potassium Alum | 0.021% | | | | | |
| Blue Pigment | 0.005% | 0.005% | 0.005% | 0.005% | | 0.008% |
| Sartomer (Urea) 511 | 1.000% | 1.000% | 3.000% | | | |
| Ciba AGEFLEX ® FA1Q80MC | 1.500% | | | | | |
| Triethanolamine | 4.000% | 5.000% | 5.000% | | | |
| Glycerin | 44.500% | 50.000% | 49.000% | 46.000% | 45.000% | 47.90% |
| Lubrizol L2405 (50% Sodium | 10.000% | 9.000% | | 16.610% | 10.000% | 10.00% |
| AMPS) | | | | | | |
| Polyvinylpyrrolidone/vinyl | | | | | 6.000% | 5.00% |
| acetate 70:30 | | | | | | |
| Noveon GoodRite ® K-732 | | | | | 1.000% | 1.00% |
| N-Vinylpyrrolidone | | | | 2.300% | 2.282% | 2.00% |
| Hydroxybutyl vinyl ether | | | | 0.550% | 0.625% | 1.50% |
| Vinylimidazole | | | | | 0.500% | 0.50% |
| COGNIS Bisomer ® PEA6 | | | | 0.300% | | |
| Diethyleneglycol monovinyl ether | 6.000% | 6.000% | 8.000% | | | |
| Ciba Irgacure ® 2959 | 0.120% | 0.150% | 0.145% | 0.135% | 0.250% | 0.25% |
| Ciba Darocur ® 1173 | | | 0.050% | | | |
| Methylene-bisacrylamide | | | | | 0.035% | 0.04% |
| Amine Acrylate 1 | 0.150% | 0.200% | 0.200% | | | |
| Amine Acrylate 2 | 0.350% | 0.400% | 0.400% | 0.375% | 0.225% | 0.225% |
| Amine Acrylate 3 | | | | | | 0.025% |
| SARTOMER SR - 9035 | 0.025% | 0.020% | 0.020% | 0.025% | | |
| Ciba Irganox ® 1010 | | 0.040% | 0.040% | 0.020% | 0.040% | 0.02% |
| 4-Methoxyphenol (MEHQ) | 0.030% | 0.150% | 0.150% | 0.100% | 0.050% | 0.10% |
| Benzophenone | 0.050% | 0.040% | 0.040% | 0.040% | 0.040% | |
| Dow Corning 193 | 0.005% | | | | | |
| Sodium Ascorbyl Phosphate | 0.250% | 0.250% | 0.250% | 0.250% | 0.250% | 0.20% |
| Polysorbate 20 | | 0.250% | 0.250% | 0.250% | 0.250% | 0.040% |
| BASF Luvitec ® VPI 55 K 72 W | 2.000% | | | 2.000% | | |
| Polyethylene Glycol 300 | 3.000% | | | 4.000% | 6.000% | 2.90% |
| Vitamin E acetate | 0.200% | | | | | |
| Acrylic Acid | 11.500% | 10.995% | 12.000% | 10.330% | 12.500% | 12.50% |
| | 100.00% | 100.00% | 100.00% | 100.00% | 100.00% | 100.00% |

The antioxidants used in these six solid hydrogel adhesive examples were MEHQ (in addition to what was already in the acrylic monomers as received), Benzophenone, Ciba Irganox 1010, Vitamin E acetate, and Sodium Ascorbyl Phosphate with Polysorbate 20 for additional aqueous stability.

These antioxidants may be used within the following ranges to obtain an electrode adhesive having suitable mechanical and adhesive properties for use as a biomedical electrode without causing erythema under normal conditions of use.

| | | |
|---|---|---|
| MEHQ | 500–1500 | ppm |
| BENZOPHENONE | 200–500 | ppm |
| IRGANOX 1010 | 200–500 | ppm |
| VITAMIN E | 200–500 | ppm |
| ACETATE | | |
| SAP | 2000–30,000 | ppm |
| POLYSORBATE 20 | 2000–30,000 | ppm |

where 0.1 pph or 0.1% = 1,000 ppm

Stimulation was carried out as in Example 1 for these electrodes. The result is shown in FIG. 4.

### EXAMPLE 9

The following oil-in-water emulsion-was prepared per BASF 62/00109 formulation example with BASF RetiSTAR (a stabilized retinol), applied prior to stimulation and allowed to dry, and gave results similar to FIG. 3.

| | % | Ingredients | INCI Name |
|---|---|---|---|
| A | 1.7 | Cremophor ® A 6 | Ceteareth-6, Stearyl Alcohol |
| | 0.7 | Cremophor ® | Ceteareth-25 |
| | | A 25 | |
| | 2.0 | Uvinul ® A Plus | Diethylamino Hydroxybenzoyl Hexyl |
| | | | Benzoate |
| | 2.0 | Abil ® B 8843 | PEG-14 Dimethicone |
| | 3.6 | Lanette ® O | Cetearyl Alcohol |
| | 6.0 | Uvinul ® MC 80 | Ethylhexyl Methoxycinnamate |
| | 2.0 | Cetiol B | Dibutyl Adipate |
| B | 5.0 | Glycerin 87% | Glycerin |
| | 0.2 | Edeta ® BD | Disodium EDTA |
| | 1.0 | D-Panthenol | Panthenol |
| | | 75 W | |
| | 68.8 | Water dem. | Aqua demineralized |
| C | 4.0 | Luvigel ® EM | Caprylic/Capric Triglyceride, Sodium |
| | | | Acrylates Copolymer |
| D | 0.2 | Sodium Ascorbyl | Sodium Ascorbyl Phosphate |
| | | Phosphate | |
| | 1.0 | Vitamin E | Tocopheryl Acetate |
| | | Acetate | |
| | 0.2 | Bisabolol rac. | Bisabolol |
| E | 0.6 | Sodium | Sodium Hydroxide |
| | | Hydroxide | |
| | | 10% aq. w/w | |
| F | 1.0 | RetiSTAR ™ | Caprylic/Capric Triglyceride Sodium |
| | | | Ascorbate Tocopherol Retinol |

Phases A and B were heated separately to about 80 C. Phase B was stirred into phase A and homogenized. Phase C was then added and homogenized. The mixture was then cooled to about 40 C then phase D was added with enough phase E to bring the pH to 6.5 with stirring. Phase F was added and the mixture homogenized then cooled to room temperature. The lotion was applied and allowed to dry. The test was carried out as in Example 1 and yielded similar results as in FIG. 3.

Although there has been hereinabove described a composition and method for reduction of skin irritation caused by electrical stimulation through use of antioxidants in accordance with the present invention for the purpose of illustrating the manner in which the invention may be used to advantage, it should be appreciated that the invention is not limited thereto. In particular, the antioxidant precursor may be incorporated in the electrode adhesive or separately applied to the skin of a patient prior to attachment of the biomedical electrode. In addition, the present invention comprises a kit including a biomedical electrode, as described above, and said antioxidant precursor in a form suitable for application to the skin of a patient and packaged for distribution, which advises a medical technician how to utilize the antioxidant precursor to prevent and/or amelliorate erythema prior to the use of said biomedical electrode to monitor and or stimulate a patient. Moreover, said invention comprises an article of manufacture which comprises 2000-30,000 ppm of Sodium Ascorbyl Phosphate. Finally, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. An electrode providing electrical contact with a patient's skin comprising:
a conductive member connectable to an external electrical apparatus; and
a substrate for electrically interfacing to said patient's skin, said interfacing substrate being electrically and mechanically connected to said conductive member, said interfacing substrate being a non-liquid water containing film and which comprises an electrically conductive organic polymer said organic polymer being derived from a monomer mixture that is polymerized in the presence of a free radical initiator wherein said monomer mixture further comprises:
(a) a catalyst that provides free radicals
and (b) an antioxidant, wherein said antioxidant is in the form of a chemical moiety which is a precursor of said antioxidant, which chemical moiety does not react with said free radicals but is converted upon contact with a patient's skin into said antioxidant.

2. The electrode of claim 1 wherein said monomer mixture comprises acrylic acid.

3. The electrode of claim 2 wherein said catalyst is a photoinitiator.

4. The electrode of claim 3 wherein said antioxidant is a vitamin.

5. The electrode of claim 4 wherein said vitamin is selected from the group consisting of Vitamins A, C, and E.

6. The electrode of claim 5 wherein said vitamin is Vitamin C.

7. The electrode of claim 1 wherein said monomer mixture further comprises from 100 to 30,000 ppm of a surfactant.

8. The electrode of claim 7 wherein said surfactant is a polyoxyethylene (20) sorbitan monolaurate.

9. The electrode of claim 1 wherein said monomer mixture further comprises from 100 to 30,000 ppm of ultraviolet sensitive curing agents.

10. The electrode of claim 9 wherein said ultraviolet sensitive curing agent is selected from the group consisting of 2-hydroxy-2-methyl-1-phenylpropan-2-one, 4-(2-hydroxyethoxy)phenyl(2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

11. A method for preventing and/or ameliorating erythema in a patient being monitored or electrically stimulated by means of a biomedical electrode which is adhered to the skin of said patient including:
said method comprising contacting said patient's skin with an antioxidant, wherein said antioxidant is in the form of a chemical moiety which is a precursor of said anti-oxidant, which chemical moiety does not react with said free radicals used to make substrate but is converted into said antioxidant upon contact with the skin of said patient.

12. The method of claim 11 wherein said electrode comprises:
a conductive member connectable to an external electrical apparatus; and
a substrate for electrically interfacing to said patient's skin, said interfacing substrate being electrically and mechanically connected to said conductive member, said interfacing substrate being a non-liquid water containing film and which comprises an electrically conductive organic polymer said organic polymer being derived from a monomer mixture that is polymerized in the presence of a free radical initiator wherein said monomer mixture further comprises a catalyst that provides free radicals.

13. The method of claim 12 wherein said monomer mixture comprises acrylic acid.

14. The method of claim 13 wherein said catalyst is a photoinitiator.

15. The method of claim 11 wherein said chemical moiety is contacted with said patient's skin prior to adhering the electrode thereto.

16. The method of claim 11 wherein said chemical moiety is incorporated in the monomer mixture.

17. The method of claim 11 wherein said antioxidant is a vitamin.

18. The method of claim 14 wherein said antioxidant is selected from the group consisting of Vitamins A, C, and E.

19. The method of claim 18 wherein said antioxidant is Vitamin C.

20. The method of claim 19 wherein said antioxidant comprises from 2000 to 30,000 ppm of said monomer mixture.

21. The method of claim 20 wherein said prodrug is sodium ascorbyl phosphate.

22. A kit including a biomedical electrode comprising:
a conductive member connectable to an external electrical apparatus; and
a substrate for electrically interfacing to said patient's skin, said interfacing substrate being electrically and mechanically connected to said conductive member, said interfacing substrate being a non-liquid water containing film and which comprises an electrically conductive organic polymer said organic polymer being derived from a monomer mixture that is polymerized in the presence of a free radical initiator wherein said monomer mixture further comprises a catalyst that provides free radicals.
and an antioxidant wherein said antioxidant is in the form of a chemical moiety converted upon contact with a patient's skin into said antioxidant,
packaged for distribution which advises a medical technician how to utilize the chemical moiety to prevent and/or ameliorate erythema during the use of said biomedical electrode to monitor and/or stimulate a patient.

23. The kit of claim 22 wherein said antioxidant precursor is in the form of a separate solution, lotion, or gel suitable for application to the skin of a patient.

24. The kit of claim 22 wherein said antioxidant is a vitamin.

25. The kit of claim 22 wherein said antioxidant is Vitamin C.

26. The kit of claim 22 wherein said antioxidant precursor is sodium ascorbyl phosphate.

27. The electrode of claim 1 wherein said monomer mixture comprises one or more of the following:
2000-30,000 ppm Sodium Ascorbyl Phosphate
200-500 ppm Vitamin E acetate
500-1500 ppm MEHQ and
200-500 ppm Benzophenone

28. The electrode of claim 27 comprising from 2000-30,000 ppm of sodium ascorbyl phosphate.

29. The electrode of claim 28 further comprising 2000-30,000 ppm Polysorbate 20.
